(19) United States
(12) Patent Application Publication
(10) Pub. No.: US 2016/0235820 A1
(43) Pub. Date: Aug. 18, 2016

(54) METHOD OF INJECTING A KERATIN-BASED MATERIAL INTO A TARGET REGION OF THE HEART

(71) Applicant: Cormend Technologies, LLC, Wellesley, MA (US)

(72) Inventor: John C. ANDRES, Chatham, MA (US)

(73) Assignee: Cormend Technologies, LLC, Wellesley, MA (US)

(21) Appl. No.: 15/045,917

(22) Filed: Feb. 17, 2016

Related U.S. Application Data
(60) Provisional application No. 62/117,869, filed on Feb. 18, 2015.

(51) Int. Cl. A61K 38/39; A61K 9/00

(52) U.S. Cl. CPC A61K 38/39; A61K 9/0019

(57) ABSTRACT

A method for direct therapeutic treatment of myocardial tissue in a localized region of a heart having a pathological condition. The method includes identifying at least one target region of the myocardium of the heart to be treated; and injecting a keratin-based material that travels to at least one target region to at least one of stiffen, restrain, or constrain the myocardial tissue of the target region. The material applied results in a physical modification of the mechanical properties, including stiffness, of the infarct tissue. Various devices and modes of practicing the method are disclosed for stiffening, restraining and constraining myocardial tissue for the treatment of conditions including myocardial infarction or mitral valve regurgitation.

### CROSS-REFERENCE TO RELATED APPLICATION

This application claims priority to U.S. Provisional Patent Application No. 62/117,869, filed Feb. 18, 2015, the disclosure of which is hereby incorporated by reference in its entirety.

### TECHNICAL FIELD

The present disclosure relates generally to therapeutic methods in the field of interventional cardiology and cardiac surgery, and more specifically to a keratin-based material to stiffen a myocardial infarction area, to shrink the myocardial infarct region, and/or to reduce wall motion in a peri-infarct and/or infarct region of a heart. The present disclosure also has application in the treatment of mitral valve regurgitation and diastolic dysfunction.

### BACKGROUND

Each year over 1.1 million Americans have a myocardial infarction, usually as a result of a heart attack. These myocardial infarctions result in an immediate depression in ventricular function and all of these infarctions are very likely to expand, provoking a cascading sequence of myocellular events known as ventricular remodeling. In many cases, this progressive myocardial infarct expansion and ventricular remodeling leads to deterioration in ventricular function and heart failure.

Post myocardial infarct drug therapy may attenuate many factors that accelerate this remodeling. More recently, medical devices have been developed which provide surgeons with limited tools to support modest intervention with respect to this remodeling situation. However, cardiologists and interventional cardiologists and cardiac surgeons presently lack any devices or procedures for directly attacking this remodeling problem.

A myocardial infarction (MI) occurs when a coronary artery becomes occluded and can no longer supply blood to the myocardial tissue. When a myocardial infarction occurs, the myocardial tissue that is no longer receiving adequate blood flow dies and is replaced with scar tissue. Within seconds of a myocardial infarction, the under-perfused myocardial cells no longer contract, leading to abnormal wall motion, high wall stresses within and surrounding the infarct, and depressed ventricular function. The infarct expansion and ventricular remodeling are caused by these high stresses at the junction between the infarcted tissue and the normal myocardium. These high stresses eventually kill or severely depress function in the still viable myocardial cells. This results in a wave of dysfunctional tissue spreading out from the original myocardial infarct region, which greatly exacerbates the nature of the disease and can often progress into advanced stages of congestive heart failure (CHF).

Infarct expansion is a fixed, permanent, disproportionate regional thinning and dilatation of the infarct zone. Infarct expansion occurs early after a myocardial infarction. The mechanism is slippage of the tissue layers.

Infarct extension is additional myocardial necrosis following myocardial infarction. Infarct extension results in an increase in total mass of infarcted tissue. Infarct extension occurs days after a myocardial infarction. The mechanism for infarct extension appears to be an imbalance in the blood supply to the peri-infarct tissue versus the increased oxygen demands on the tissue.

Ventricular remodeling is progressive enlargement of the ventricle with depression of ventricular function. Myocyte function in the myocardium remote from the initial myocardial infarction becomes depressed. Ventricular remodeling usually occurs weeks to years after myocardial infarction. There are many potential mechanisms for ventricular remodeling, but it is generally believed that the high stress on peri-infarct tissue plays an important role. Due to altered geometry, wall stresses are much higher than normal in the myocardial tissue surrounding the infarction.

High wall stress can directly damage myocytes. While there are other potential mechanisms, applicant recognizes that the skeletal muscle literature suggests that the high wall stress can lead to cellular dysfunction and damage. This mechanism has been disclosed in U.S. Pat. Nos. 8,936,027; 8,452,419; and 7,988,727, the disclosures of which are hereby incorporated by reference.

The skeletal muscle literature suggests that muscle tissue can be acutely injured by high stress. Applicant realizes that stress-induced injury can also occur in cardiac muscle subjected to repeated high stress contractions which occur along the progressive boundaries of an initially-infarcted tissue site. High stresses in the peri-infarct region results in the death or dysfunction of otherwise viable tissue, resulting in a progressive increase in the size of damaged tissue. As new tissue is continuously subjected to high stresses the tissue adjacent to it dies or becomes dysfunctional and results in a new, enlarged peri-infarct region.

### DESCRIPTION OF RELATED ART

Immediately after a myocardial infarction, preventing and treating ventricular fibrillation and stabilizing the hemodynamics are well-established therapies. Newer approaches include more aggressive efforts to restore patency to occluded vessels. This is accomplished through thrombolytic therapy or angioplasty and stents. Reopening the occluded artery within hours of initial occlusion can decrease tissue death, and thereby decrease the total magnitude of infarct expansion, extension, and ventricular remodeling.

Chronic treatments include surgical approaches to exclude, isolate, or remove the infarct region (such as the Dor procedure). Other potential surgical approaches require the chest to be opened, such as with the CARDIOCAP made by Acorn Cardiovascular Inc. of St. Paul, Minn. The CARDIOCAP device, a textile girdle or so-called “cardiac wrap,” is wrapped around both the left and right ventricles, thereby preventing further enlargement of the heart. The treatment can include the application of heat to shrink the infarcted, scarred tissue, followed by the suturing of a patch onto the infarcted region. Other treatments envision surrounding the heart, or a significant portion thereof, with a jacket.

Chronic treatments also include pharmaceuticals such as ACE inhibitors, beta blockers, diuretics, and Ca++ antagonists. These agents have multiple effects, but share in the ability to reduce aortic pressure, and thereby cause a slight decrease in wall stress and decrease ventricular remodeling. However, drug compliance is far from optimal. Significant variances exist between published guidelines and actual practice.

Cellular transplantation, introduction of cells into terminally injured heart, can mediate over several weeks into islands of viable cells in the myocardium. Several different cell types, ranging from embryonic stem cells, smooth muscle cells, bone marrow cells, cardiomyocytes to autologous skeletal myoblasts have been successfully propagated within damaged heart and shown to improve myocardial performance.

A catheter-based approach for the introduction of devices or agents into the cardiac space can take advantage of current techniques to identify the ischemic myocardium, to position catheters within the left ventricular cavity, to insert devices onto or into the myocardium, and/or to inject material into a coronary artery or vein.

Multiple technologies and approaches are available today for the clinician to assess normal, ischemic-non-viable, and ischemic-viable myocardial tissue. These include, but are limited to, localized blood flow determinations, local electrical and mechanical activity, nuclear cardiology, echocardiographic stress test, coronary angiography and ventriculography.

The present disclosure contemplates an intervention being taken to prevent further deterioration of cardiac tissue surrounding an infarction, such intervention being taken soon after or immediately following an MI event. This is achieved by supporting an infarct and/or peri-infarct region to prevent processes associated with infarct expansion and ventricular remodeling as the result of high stresses exerted at the junction between the infarcted tissue and the normal myocardium.

Proposals have been made to inject various compositions into the infarct region or surrounding tissue to stiffen, constrain or restrain the infracted area. See Santamore U.S. Pat. No. 8,936,027; Gorman published U.S. patent application 2012/0148630; Cohen published US patent application 2006/0083721 and Sabbah U.S. Pat. No. 8,419,711, all the disclosures of which are hereby incorporated by reference. The present disclosure relates to further improvements to such treatments.

### SUMMARY

In an aspect, there is disclosed a method for direct localized therapeutic treatment of myocardial tissue in a heart, the method comprising the steps of identifying at least one target region of the myocardium of the heart to be treated; and injecting a keratin-based material that travels to the at least one target region to at least one of stiffen, restrain, or constrain the target region of the myocardium.

Additional objects and advantages of the invention will be set forth in part in the description which follows, and in part will be obvious from the description, or may be learned by practice of the materials and methods of the present disclosure. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DESCRIPTION OF ILLUSTRATIVE EMBODIMENTS

Reference will now be made in detail to exemplary embodiments of the disclosure.

As discussed hereinabove, within seconds of a myocardial infarction underperfused myocardial cells no longer contract, and actually lengthen during systole leading to abnormal wall motion, high wall stresses within and surrounding the infarct, and bulging in the ischemic regions. The present invention serves to reduce the abnormal geometry and wall stress placed on the peri-infarct and/or infarct tissue. As a general proposition, materials and methods of the present disclosure limit motion in the peri-infarct and/or infarct region. Thereby, the infarct region is made stiffer, altering the geometry and wall stress on the peri-infarct tissue. Thereby, the infarct region is effectively excluded or shrunk. In another, aspect, the present disclosure has as one of its objects to restrict motion in the peri-infarct and/or tissue; i.e., to eliminate or limit expansion of the peri-infarct tissue during systole. Otherwise, the still-viable myocardial cells must shorten more than normal to compensate for this wasted, abnormal wall motion in the infarct region, and further, such extra shortening occurs against a higher wall stresses.

According to the present disclosure, the keratin-based materials may be pure keratin, purely keratin derived materials, mixtures of keratin materials or keratin derived materials, mixtures, emulsions or blends of keratin or keratin derived materials with non-keratin materials, or block or random copolymers of keratin, keratin derived materials or keratin containing materials.

According to the present disclosure, regional passive tissue characteristics can be altered, for example, by three approaches that will be discussed herein; stiffening, restraining, or constraining. If the tissue is stiffened, the length of the tissue changes little, if at all, over a very wide range of tension; i.e., the length is almost independent of the pressure in the ventricle. If the tissue is restrained, the normal exponential relationship between tension and length exists until the tissue reaches an upper length limit. At this length, any further lengthening of the tissue is prevented and the tension-length relationship becomes almost a straight line. The length of the tissue changes little, if at all, above this length. If the tissue is constrained, at every length, a higher tension is required to stretch the tissue to that length.

There is disclosed herein a method for direct localized therapeutic treatment of myocardial tissue in a heart. The method comprises identifying at least one target region of myocardium of the heart to be treated; and injecting a keratin-based material into the at least one target region to at least one of stiffen, restrain, or constrain the target region.

As discussed above, methods for identifying target regions of the heart are known and are well within the skill of one of ordinary skill in the art. Non-limiting examples include, localized blood flow determinations, local electrical and mechanical activity, nuclear cardiology, echocardiographic stress test, coronary angiography and ventriculography.

The infarcted tissue must be identified and located on the heart. There are many clinical means known in the art to identify and locate infarcted heart tissue. The occluded coronary artery that caused the myocardial infarction is also identified using known methods. The occluded artery, the region of the heart perfused by this artery, and thus the infarcted tissue, are naturally related.

Further, infarcted heart tissue has unique characteristics: no or minimal electrical activity, different electrical impedance properties, abnormal wall motion, and abnormal metabolic activity. Each of these is used individually or in combination to identify the infarcted tissue. In one approach, a catheter(s) deployed in the left ventricle has electrodes at its tip. By positioning the catheter(s) against the left ventricular endocardial border and recording the local electrical activity, infarcted tissue is recognized (i.e., through observing very low electrical potentials) (Callans, D. J. et al., “Electroanatomic Left Ventricular Mapping In The Porcine Model Of Healed Anterior Myocardial Infarction: Correlation With Intracardiac Echocardiography And Pathological Analysis,” Circulation 1999; 100:1744-1750). In another approach, the catheter has several small electrodes by its tip. These electrodes measure the local electrical impedance of the tissue by the catheter's tip. Infarcted myocardial tissue impedance is significantly lower than the impedance of normal myocardial tissue (Schwartzman D. et al., “Electrical Impedance Properties Of Normal And Chronically Infarcted Left Ventricular Myocardium,” J. Intl. Cardiac Electrophys. 1999; 3:213-224; Cinca J. et al., “Passive Transmission Of Ischemic ST Segment Changes In Low Electrical Resistance Myocardial Infarct Scar In The Pig,” Cardiovascular Research 1998; 40:103-112). Again, these approaches can be combined: the same electrodes that measure local electrical activity also measure local electrical impedance. The contents of these publications are incorporated herein by reference.

The disclosed method contemplates that more than one, i.e., at least one, target region of the myocardium may need to be identified and/or treated. Non-limiting examples of the at least one target region include the peri-infarct and infarct tissue of the myocardium.

The disclosed method further comprises injecting a material into the at least one target region. The material is a keratin-based material. Generally speaking, keratins are animal or mammal scleroprotein or album inoid substances found in the dead outer skin layer, in horns, hair, feathers, hoofs, nails, claws, bills, etc. The keratin-based materials contemplated and described herein may be made from or derived from processed animal parts containing keratin, or may be synthesized based on such materials or to mimic the composition of such materials, including by recombinant production of proteins identified from or derived from keratin.

As used herein, the term “keratin-based material” includes any material comprising any amount of keratin in a native and a non-native form, pure keratin, processed keratin, keratin mixtures, keratin blends, polymers or copolymers containing keratin, emulsions containing keratin, keratin hydrogels, or any of the foregoing containing keratin derived materials such as recombinantly or synthetically made keratin materials or keratin proteins. As an example, the keratin-based material may be a keratin protein fraction. Keratin protein fractions are distinct groups from within the keratin protein family, such as the intermediate filament proteins, the high sulfur proteins or the high glycine-tyrosine proteins well known in the art. Intermediate filament proteins are described in detail by Orwin et al (Structure and Biochemistry of Mammalian Hard Keratin, Electron Microscopy Reviews, 4, 47, 1991) and also referred to as low sulphur proteins by Gilliespie (Biochemistry and physiology of the skin, vol 1, Ed. Goldsmith Oxford University Press, London, 1983, pp 475-510). Key characteristics of this protein family are molecular weight in the range 40-60 kD, and a cysteine content (measured as half cystine) of around 4%. The high sulfur protein family is also well described by Orwin and Gillespie in the same publications. This protein family has a large degree of heterogeneity but can be characterized as having a molecular weight in the range 10-30 kD, and a cysteine content of greater than 10%. The subset of this family, the ultra-high sulfur proteins can have a cysteine content of up to 34%. The high glycine-tyrosine protein family is also well described by Orwin and Gillespie in the same publications. This family is also referred to as the high tyrosine proteins and has characteristics of a molecular weight less than 10 kD, tyrosine content typically greater than 10% and a glycine content typically greater than 20%. In preferred embodiments, the keratin-based material is predominately composed of keratin, that is, a majority of the material is keratin or a keratin derived material. More preferably, the keratin or keratin derived material included in the keratin-based material is substantially pure (i.e., 95% or greater content by weight) of keratin or a keratin derived material.

For the purpose of this disclosure, a “keratin protein fraction” is a purified form of keratin that contains predominantly, although not entirely, one distinct protein group as described above. As an example, S-Sulfonated keratins have cysteine/cystine present predominantly in the form S-sulfocysteine, commonly known as the Bunte salt. This highly polar group imparts a degree of solubility to proteins. Whilst being stable in solution, the S-sulfo group is a labile cysteine derivative, highly reactive towards thiols, such as cysteine, and other reducing agents. Reaction with reducing agents leads to conversion of the S-sulfo cysteine group back to cysteine. S-sulfo cysteine is chemically different to cysteic acid, although both groups contain the SO₃ group. Cysteic acid is produced irreversibly by the oxidation of cysteine or cystine and once formed cannot form disulfide crosslinks back to cysteine. S-sulfocysteine is reactive towards cysteine and readily forms disulfide crosslinks.

The keratin-based material can be in the form of a hydrogel, fiber, film, or membrane. Any of the foregoing may be solubilized for ease of handling, such as delivery though a syringe, needle, catheter or the like. In addition, the keratin-based materials may be combined with other substances or formed into specific shapes and sized particles, or may be formed to contain other substances.

In an aspect, the keratin-based material can be a hydrogel. For example, U.S. Pat. No. 6,379,690, the disclosure of which is hereby incorporated by reference, discloses how to make a keratin hydrogel. For example, an oxidizing agent is added to a keratin solution to crosslink the keratin proteins. Non-limiting exemplary oxidizing agents include hydrogen peroxide, organic peracids, peroxy carbonates, ammonium sulfate peroxide, benzoyl peroxide, and perborates. Hydrogen peroxide, for example, can be added to the keratin solution at about 0.5% to about 1.0% w/v, mixed well, and allowed to stand at room temperature for several days, such as, for example, about 3 days. The freely flowing solution slowly thickens and converts to a cross-linked hydrogel after about 72 hours.

In another aspect, the keratin-based material can be a fiber. Keratin-based fibers are prepared through the intimate mixing of a keratin solution with a water soluble polymer, such as PVA or PVP, followed by extrusion into an appropriate coagulation solution in which both components are insoluble. Subsequent strengthening of the composite fibers occurs by introducing a cross-linking reagent or heat treatment in order to raise the crystallinity of the materials through removal of residual water and the formation of new hydrogen bond between the molecules.

In another aspect, the keratin-based material can be a film or membrane. Composite films or membranes can be formed from S-sulfonated keratin protein fractions. Intimately mixed solutions of S-sulfonated keratin intermediate filament proteins (SIFP) (for example 5%) and water soluble polymers such as, polyvinyl alcohol (PVA), polyvinyl pyrrolidone (PVP) (up to 10% solids) are cast and the solution solvents evaporated to leave a keratin-polymer composite film or membrane. The solvent can be aqueous based and include some percentage of organic based aqueous miscible solvent, such as an alcohol.

The physical and mechanical properties of the composite materials can be readily improved through a variety of methods known to those of ordinary skill in the art and disclosed in U.S. Pat. No. 7,767,756. In addition, the toughness or strength of the keratin-based material can be increased by standard protein cross-linking methods including using, typical chemical cross-linkers such as, glutaraldehyde, formaldehyde, carbodiim ides, e.g., 1-ethyl-3-(dimethylaminopropyl)carbodiimide, 2,5-hexanedione, diimidates, e.g., dimethylsuberimidate, or bisacrylamides, e.g., N,N′-methylenebisacrylamide.

Keratin derivatives can be synthesized which involve chemical bonds forming between the keratin proteins and synthetic monomers; such as those from the vinyl family including, acrylates and epoxy acrylates-based monomers. Methods for synthesizing keratin derivatives can be found in U.S. Pat. No. 7,767,756, the disclosure of which is hereby incorporated by reference. Composite materials formed in this way may then be further processed, either through dry, wet or melt extrusion techniques into films or membranes, fibers and other materials, such as thermoplastic or thermoset materials These materials can be processed through single or multi extruding or compression molding techniques. The mechanical properties of the synthetic polymer component may be modified by inclusion of suitable co-monomers to allow low-temperature thermoforming processes in which the integrity of the protein component is kept intact.

Methods of controlling the solubility of the keratin-based material can be found in U.S. Pat. No. 7,001,988, the disclosure of which is hereby incorporated by reference. Additionally, various isolated peptides of keratin can be found in U.S. Pat. No. 7,501,485, the disclosure of which is hereby incorporated by reference. U.S .Pat. No. 7,001,987, discloses compositions comprising water soluble keratin proteins of a certain molecular weight wherein the composition forms a hydrogel when placed in an aqueous ion containing solution, the disclosure of which is hereby incorporated by reference.

In another aspect, the keratin-based material for use in the disclosed method can include materials selected from the group consisting of metal particles in a viscous biocompatible gel matrix; two or more biocompatible polymer precursors; liquid plastic materials; collagen; non-absorbable and/or biocompatible materials; and combinations thereof.

The keratin-based material may include metal particles in a viscous biocompatible gel matrix, which are injected into the target region. The infarct tissue stiffens due to the properties of the keratin-based material injected and also due to encapsulation of the material by the body. Additionally, these encapsulated areas tend biologically to link together, further stiffening the myocardium.

In a further embodiment, the keratin-based material can include two or more biocompatible polymer precursors, such as hydrogels, which when mixed increase in viscosity and/or stiffness, which when injected in the infarcted region, serve to stiffen it. The precursors can be mixed prior to injection, or mixed in situ. The injected or perfused precursors can contain additional particulates for stiffening, or be injected without. The injected keratin-based material can also include luminescent, radiopaque or other contrast agents to enhance visualization.

In another embodiment, liquid plastic materials containing keratin-based material are used. The liquid plastic material containing keratin-based material is injected into the coronary vein draining the infarct tissue. The material solidifies, thereby stiffening the infarct tissue.

In another aspect, the keratin-based material includes collagen, a safe material that appears inert and has minimal incidence of adverse effects. Collagen is a naturally occurring protein found in skin, bone, and connective tissue. It breaks down and is excreted over time.

The keratin-based material itself can be non-absorbable. Such material is biocompatible, but is not absorbable to the extent that injection or perfusion of the material into the infarcted region leads to encapsulation. Many materials can be used with the keratin-based material, such as metal filings. In another embodiment, non-metallic materials are used, including various plastics. Materials that readily absorb different types of energy such as ultrasound and/or microwaves can also be used. Representative materials include metals (e.g., Stainless Steel, Titanium, Nitinol), nonmetals and polymers (e.g., Carbon, including Pyrolytic Carbon, Teflon, Polymers, Silicone, Polyurethane, Latex, Polypropylene, Epoxy, Acrylic, Polycarbonate, Polysulfone, PVC), fibrous materials (e.g., Polyester, ePTFE, Teflon Felt), and natural substances (e.g., Starch, Cat Gut). Of course, this list is merely exemplary and any biocompatible material can be used.

Materials contemplated for use with keratin-based materials in the practice of the present invention include biodegradable polymers, polylactide (PLA), polyglycolide (PGA), lactide-glycolide copolymers (PLG), polycaprolactone, lactide-caprolactone copolymers, polyhydroxybutyrate, polyalkylcyanoacrylates, polyanhydrides, polyorthoesters, proteins such as albumin, collagen, gelatin, polysaccharides such as dextrans, starches, biocompatible polymers contemplated include acrylate polymers and copolymers; methyl methacrylate, methacrylic acid; hydroxyalkyl acrylates and methacrylates; methylene glycol dimethacrylate; acrylamide, bisacrylamide; cellulose-based polymers; ethylene glycol polymers and copolymers; oxyethylene and oxypropylene polymers; poly(vinyl alcohol) and polyvinyl acetate; polyvinylpyrrolidone and polyvinylpyridine.

The keratin-based material is placed either through a percutaneous, mini-thoracotomy, or open chest approach wherein the material is directly injected into the target region. The target region is located as previously described.

For the percutaneous approach, a catheter is placed in the left ventricle of the heart and positioned against the endocardial border. In one embodiment, a guidewire with side holes and a lumen is advanced into the infarcted tissue. Once within the infarcted tissue, the keratin-based material is injected. These injections are repeated in multiple regions of the infarct.

For an open chest approach, a small needle can be inserted into the infarct tissue and the keratin-based material is injected. A similar procedure is used for a mini-thoracotomy approach.

In another delivery approach, the keratin-based material is injected into a coronary artery or vein to reach the infarcted tissue. The advantage to this approach is that larger particles can be injected into the venous system without affecting coronary blood flow. In this approach, a guide catheter is positioned in the coronary sinus via a vein, such as the right or left femoral vein. The guide catheter is advanced into the great cardiac vein, and a smaller catheter positioned in the coronary vein in the infarct region. A guidewire is used to assist this placement. In one approach, this catheter is similar to a balloon occlusion catheter; the catheter has a central lumen and an external balloon that is inflated thereby occluding the coronary vein. Once occluded, the keratin-based material is injected retrograde into the coronary vein. The material may have barbs, shapes, or coatings that facilitate embedding or entrapment in the small veins and capillaries. This leaves the keratin-based material in the infarct tissue region. Methods for injecting material through the coronary venous system are disclosed in U.S. Pat. Nos. 7,311,731; 7,988,727; and 8,936,027, the disclosures of which are hereby incorporated by reference.

In another embodiment, the material is injected intravenously, travels through the circulation to the infarct tissue, and fixes the target tissue.

The present invention also contemplates implanting devices and/or microspheres into the myocardial tissue to accomplish stiffening, restraint or constraint of the tissue. In an aspect, the injection of the disclosed keratin-based material can be used in conjunction with the implantation of devices.

Various types of implantable devices and methods of inserting those into the target region can be found in U.S. Pat. Nos. 7,311,731; 7,988,727; and 8,936,027, the disclosures of all of which are hereby incorporated by reference.

In practice, the devices can be placed either during a percutaneous, minithoracotomy, or during an open chest approach. In the percutaneous approach, as discussed above, a catheter is introduced into a blood vessel, such as the left or right femoral artery, and advanced into the heart, for example the left ventricle. An exemplary device which could be adapted in the practice of the present invention is disclosed in U.S. Pat. No. 6,071,292 to Makower, specifically in FIGS. 7 through 14 thereof. The entirety of the Makower patent is incorporated herein by reference.

Microspheres, as known in the art, may be applied with a keratin-based material through a variety of techniques, for example injection into blood stream or tissue, open surgical and minimally invasive implantation. One such approach is to inject metal microspheres into the infarcted myocardium. This method is disclosed in U.S. Pat. Nos. 7,311,731; 7,988,727; and 8,936,027, the disclosures of which are hereby incorporated by reference.

Also contemplated is a bisphere configuration: a layer or shell within a shell. The inner shell, formed from biodegradable biopolymers may include keratin-based material, and provides physical structure and controls acoustic response, while the outer layer functions as the biological interface and provides a scaffold for site-specific targeting ligands. Each layer or shell can be independently modified to fulfill specific application requirements. The core or payload space can be filled with a gas such as nitrogen for ultrasound imaging such as a myocardial perfusion agent or with biotherapeutic agents for drug delivery applications.

The dual shelled microspheres containing keratin-based material can be designed to hold a variety of drugs or biotherapeutic agents. These are lyophilized and reconstituted prior to intravenous injection. The bispheres circulate through the blood stream and can be visualized using standard ultrasound diagnostic imaging instrumentation. The bispheres can be fractured by insonation with a special ultrasound “bursting” signal focused on a target site. The collapse of fracturing bispheres within the target site can be acoustically detected providing feedback as to the quantity of active drug being released at the site. The use of bispheres to transport agents to specific sites within the body can substantially increase local efficacy while decreasing systemic side effects or adverse reactions.

Microspheres advantageously allow for controlled-release (extended-release and time-release) delivery systems, and targeted and site-specific delivery systems. Microspheres advantageously can be made from expandable and/or dissolvable material preferably containing keratin-based material. They are proven able to be encapsulated, from diverse therapies using bulking agents, cyano, drug therapy, and peptides

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for direct localized therapeutic treatment of myocardial tissue in a heart, the method comprising the steps of:
identifying at least one target region of the myocardium of the heart to be treated;
injecting a keratin-based material that travels to the at least one target region to at least one of stiffen, restrain, or constrain the target region of the myocardium.

2. The method of claim 1, wherein the step of identifying at least one target region comprises identifying an infarct region.

3. The method of claim of claim 1, wherein the at least one target region comprises at least one of a peri-infarct region and an infarct region.

4. The method of claim 1, wherein the keratin-based material is in a native form.

5. The method of claim 1, wherein the keratin-based material is in a non-native form.

6. The method of claim 1, wherein the keratin-based material is in the form chosen from a hydrogel, a fiber, a film, or a membrane.

7. The method of claim 1, wherein the keratin-based material is a crosslinked hydrogel.

8. The method of claim 1, wherein the keratin-based material is a fiber comprising a water-soluble polymer.

9. The method of claim 8, wherein the water-soluble polymer is selected from the group consisting of polyvinyl alcohol or polyvinyl pyrrolidone.

10. The method of claim 1, wherein the keratin-based material is a film formed from S-sulfonated keratin protein fractions.

11. The method of claim 11, wherein the film comprises a water soluble polymer.

12. The method of claim 1, wherein keratin-based material includes material selected from the group consisting metal particles in a viscous biocompatible gel matrix; two or more biocompatible polymer precursors; liquid plastic materials; collagen; non-absorbable and/or biocompatible materials; and combinations thereof.

13. The method of claim 1, wherein the keratin-based material becomes encapsulated in the at least one of the target region.

14. The method of claim 1, further comprising the step of accessing the heart to inject the keratin-based material through an approach selected from the group consisting of an open chest approach, a mini-thoracotomy approach and a percutaneous approach.

15. The method of claim 1, wherein the step of injecting further comprises injecting a luminescent, radiopaque or other contrast agent to enhance visualization.

16. The method of claim 1, wherein the step of injecting comprises intravenously injecting the keratin-based material such that the keratin-based material travels through the circulation to the at least one target region.
